(19) United States
(12) Patent Application Publication
(10) Pub. No.: US 2021/0076944 A1
(43) Pub. Date: Mar. 18, 2021

(54) SYSTEM AND METHOD FOR NON-CONTACT ULTRASOUND IMAGE RECONSTRUCTION

(71) Applicant: Massachusetts Institute of Technology, Cambridge, MA (US)

(72) Inventors: Brian Anthony, Cambridge, MA (US); Xiang Zhang, Cambridge, MA (US); Jonathan Randall Fincke, Lincoln, MA (US); Robert W. Haupt, Lexington, MA (US); Charles M. Wynn, Groton, MA (US)

(21) Appl. No.: 17/025,309

(22) Filed: Sep. 18, 2020

Related U.S. Application Data
(60) Provisional application No. 62/902,130, filed on Sep. 18, 2019.

(51) Int. Cl. A61B 5/00; G06T 11/00

(52) U.S. Cl. CPC A61B 5/0095; G06T 2210/41; G06T 11/005; A61B 5/0077

(57) ABSTRACT

A system and method for image reconstruction for non-contact ultrasound is provided, where maps or ultrasound images of a subject may be generated without physically contacting the subject. Adjusting or optimizing the photoacoustic excitation system may be performed, such as with beam shaping, surface modifications, or closed-loop automated adjustments. 2D and/or 3D spatial locations of source and receiver laser spots may be used to provide a spatial reference location for ultrasound image reconstruction in a clinically efficacious manner. In addition, point tracking, surface profile characterization, laser adjustments, and/or surface enhancements may be used to facilitate image reconstruction of the subject.

### CROSS-REFERENCE TO RELATED APPLICATIONS

This application is based on, claims priority to, and incorporates herein by reference in its entirety, U.S. Provisional Application Ser. No. 62/902,130, filed Sep. 18, 2019.

### STATEMENT REGARDING FEDERALLY SPONSORED RESEARCH

N/A

### BACKGROUND

The present disclosure relates to systems and methods for generating and using ultrasound data, and more particularly to systems and methods for generating ultrasound images without system contact to a subject, which may be achieved, for example, using photoacoustic energy and/or laser vibrometry, in a manner that is clinically efficacious and allows for image reconstructions.

Acoustic energy is used in numerous applications to characterize discontinuities, defects, and other mechanical properties within various types of materials. Acoustically-based techniques rely on differences in mechanical properties between a feature of interest and its local surroundings. These differences result in different vibrational responses to sonic excitation, which may be detected and the feature thereby localized and/or characterized.

An important advantage of acoustic techniques is the ability to detect discontinuities corresponding to (or indicating the presence of) flaws or hidden items that may not be detectable using visual or other techniques. Such discontinuities may represent latent defects that can compromise the mechanical integrity of load-bearing structures. Coupling a sufficient amount of acoustic energy into the medium of interest is critical, and in many cases it is the factor most limiting the efficacy of the technique. The large impedance mismatch between the ambient air and most solids or liquids makes the transfer of acoustic energy into the medium a generally inefficient process. Loudspeakers are omnidirectional thus suffering significant losses at large ranges. Parametric acoustic array (PAA) sources can provide directionality, but are limited to ranges on the order of tens of meters. An efficient means of coupling acoustic energy into media could have wide ranging benefits.

Ultrasonic imaging of the internal details of the human body can provide advantages relative to other techniques such as X rays (soft tissue contrast) and MRIs (faster and less cumbersome data acquisition). Typically, ultrasonic imagery is obtained in a “contact” manner in which an ultrasonic transducer (which both sends and receives the acoustic signals) is placed directly on (i.e., in contact with) the area of interest. Because of the very large acoustic impedance mismatch between air and the human body (acoustic coupling efficiency is only about 10-3), a coupling gel is generally used at the interface between transducer and tissue to increase coupling efficiency into the body. A range of contact ultrasound techniques exist. In general, an acoustic pulse is emitted into the body. Echoes from structures are reflected back to the transducer, with the time of arrival giving information about the range to the structure. In a simple but fairly standard incarnation, the acoustic source is omnidirectional, thus only range information is obtained. A two-dimensional image is formed by using a line of transducers, which yield information in the cross range direction.

In certain circumstances, noncontact operation is highly desirable. There exist surgical situations in which sterility is an issue, where remote patient monitoring may be required, such as remote neonatal monitoring, situations in which contact is unpleasant or painful (such as imaging the eye or sensitive skin regions), or emergency situations in which the patient is in transit and/or being stabilized and may not be easily imaged via a contact system. In certain triage situations it may be desirable to image multiple patients in as rapid a manner as possible, and a noncontact system may be able to provide this capability. Additional applications include, real-time surgical feedback imaging, traumatic brain injury (TBI) detection, bone health monitoring, and others. For example, real-time surgical guidance and feedback would greatly improve from an imaging technique that can directly access exposed skin or traumatized tissue without contact, especially in very delicate procedures such as spinal and neck surgery.

Using a laser ultrasound system enables remote noncontact imaging and eliminates coupling gels (used in conventional ultrasound) applied on skin that can contaminate open body tissues. In addition, a laser system can provide fine spatial and temporal resolution to yield high quality images while reducing distortion observed with contact sensing deformation. Other benefits of such a system include minimizing patient discomfort over injured areas and setup times to acquire images, and such a system is unlikely to interfere with other methods such as MRI, CT scan, fluoroscope, etc. A portable, lightweight non-contact ultrasonic vibration imaging device can provide very significant advantages over traditional ultrasonic contact devices. Ideally, a low power handheld laser imaging system can be used not only in a hospital setting, but would provide tremendous benefits in field operations. However, existing photoacoustic systems are limited in imaging depth capability and require a transducer to be in contact with a patient and act as a receiver.

Considerable work is also ongoing to form three-dimensional images via the scanning of the transducer line array. However, this presents registration error challenges as the individual 2D images must be aligned properly. Investigations are also ongoing to combine the individual source elements of 2D arrays of transducers in such a way that the transmitted energy has a directionality to obtain better quality spatial information. However, practical implementation of 2D arrays suffer from challenges in making a sufficiently large array that is conformal and uniformly coupled to the surface of interest (e.g. the human body). Noncontact systems have the potential to mitigate the problems mentioned above related to 2D arrays if used in conjunction with a remote array source.

Thus, there is a need for systems and methods capable of providing an efficient means for coupling acoustic energy into media in a noncontact manner to generate ultrasound images in a manner that allows for image reconstruction in a clinically efficacious manner.

### SUMMARY OF THE DISCLOSURE

The present disclosure addresses the aforementioned drawbacks by providing a system and method for non-contact ultrasound that provides for image reconstruction in a clinically efficacious manner. 2D and/or 3D spatial locations of source and receiver laser spots may be used to provide a spatial reference location for ultrasound image reconstruction. In some configurations, point tracking, surface profile characterization, laser adjustments, and/or surface enhancements may be used to facilitate 3D image reconstruction of a subject.

In one configuration, a method is provided for generating ultrasound images of a subject. The method includes directing a photoacoustic excitation source to transmit acoustic energy onto a surface of a subject to induce propagating photoacoustic waves and determining a location of the transmitted acoustic energy on the surface of the subject. The method also includes translating acoustic energy along the subject to generate at least one resultant photoacoustic wave within the subject. Vibrations created by backscatter of the least one resultant wave from the structures within the subject are detected with a sensor at the surface of the subject. A location of the detected vibrations is determined at the surface of the subject. Ultrasound images are generated of the structures within the subject using the vibrations detected, the location of the transmitted acoustic energy, and the location of the detected vibrations at the surface of the subject.

In one configuration, a system is provided for generating ultrasound images of a subject. The system includes a photoacoustic excitation source configured to transmit acoustic energy onto a surface of a subject to induce propagating photoacoustic waves. The system also includes a sensor configured to detect vibrations at the surface of the subject created by backscatter of at least one resultant wave from structures within the subject. The system also includes a computer system configured to: i) determine a location of the transmitted acoustic energy on the surface of the subject; ii) translate the acoustic energy along the subject to generate at least one resultant photoacoustic wave within the subject; iii) determine a location of the detected vibrations at the surface of the subject; and iv) generate ultrasound images of the structures within the subject using the vibrations detected, the location of the transmitted acoustic energy, and the location of the detected vibrations at the surface of the subject.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a schematic diagram showing a system configured to implement the present invention for generating ultrasound images.

FIG. 2 is an image showing an excitation source and sensing array configured to be implemented into the system of FIG. 1.

FIG. 3A is a diagram showing an excitation source to be implemented into the system of FIG. 1.

FIG. 3B is a diagram showing another excitation source to be implemented into the system of FIG. 1.

FIG. 4 is a diagram showing a laser Doppler vibrometer system configured to be implemented into the system of FIG. 1.

FIG. 5 is a schematic diagram showing a system configured to implement the present invention for generating ultrasound images.

FIG. 6 is a flowchart depicting non-limiting example steps for 3D image reconstruction of a target in accordance with the present disclosure.

FIG. 7 is another flowchart depicting non-limiting example steps for 3D image reconstruction of a target in accordance with the present disclosure.

FIG. 8 is a schematic diagram showing a system configured to implement the present invention for generating ultrasound images using surface enhancements.

FIG. 9 is yet another flowchart depicting non-limiting example steps for 3D image reconstruction of a target in accordance with the present disclosure.

FIG. 10 is a non-limiting example cross section for a source laser beam incident upon a tissue surface.

FIG. 11 is a flowchart of non-limiting example steps for generating propagating photoacoustic waves in a subject.

FIG. 12 is another flowchart of non-limiting example steps for generating propagating photoacoustic waves in a subject.

### DETAILED DESCRIPTION

As will be described, the present disclosure includes a variety of systems and methods that may be used alone or in combination. In one configuration, a system is provided for using photoacoustic excitation phenomena to generate propagating elastic waves into the body that can then reflect, refract, scatter, and absorb off interior structures. In this regard, a non-contact photoacoustic excitation source is provided that can steer the ultrasonic elastic wave beam as desired into the body. These elastic waves then propagate back to the skin surface, where they are measured and used to facilitate analysis of the body. In some configurations, systems and methods are provided for adjusting or optimizing the photoacoustic excitation system, such as with beam shaping, surface modifications, closed loop adjustments, and the like to optimize photoacoustic coupling in the subject, improve image quality, reduce noise, adjust the focusing depth, to optimize time delays, increase signal, address surface reflectivity, address surface orientation, and the like. Another component described hereafter includes determining 2D and/or 3D spatial locations of source and receiver laser spots, which may be used to provide a spatial reference location for ultrasound image reconstruction. In some configurations, point tracking, surface profile characterization, laser adjustments, and surface enhancements may be used to facilitate image reconstruction of a subject.

Laser ultrasound (LUS) may be used in accordance with the present disclosure for medical imaging, 3D external and internal volume reconstructions, non-destructive testing of non-uniform surface geometries, and the like. LUS may provide for zero image loading, does not require a gel or surface treatment as with conventional ultrasound, has minimal interference, does not promote repetitive user strain as does conventional ultrasound, and may reduce the risk of infection in a subject. In one configuration, LUS employs photoacoustic sources in combination with laser interferometric detection. LUS for medical imaging can be applied for non-contact ultrasound imaging, elastography, imaging of sensitive of painful tissue regions, live imaging during surgeries, residual limb imaging for improved prosthetic fittings without applying load to the limb, and the like. LUS may see high usage in critical care situations where whole organ and high temporal resolution imaging is often needed but forgone due to radiation dosage concerns of CT and inability to safely put patients into MRI scanners.

A LUS source, such as a laser or a plurality of lasers, may be directed on the subject and used for both generation and detection of sound. LUS sound generation may be achieved via short optical pulses that generate ultrasonic waves at a surface of a subject, such as the skin surface. Short pulses of optical energy are converted into mechanical energy via thermoelastic expansion and relaxation causing the optically absorbing region to rapidly deform, thus launching a propagating mechanical wave. LUS sound reception is achieved by measuring the returning waves at the skin surface with a laser Doppler vibrometer. Imperfect estimates of sound source and detector position are known from the system geometry

LUS has been described in US 20170265751A1, US 20170258332A1, and US 20150148655A1, all of which are hereby incorporated by reference in their entirety. Laser ultrasound (LUS) has been recently demonstrated as a viable method to replace traditional piezoelectric contact transducers for ultrasound imaging. LUS methods have the distinct advantages of non-contact, large area, and operator-free ultrasound imaging. LUS may include emission of a pulsed laser source upon a biological tissue surface that generates a localized acoustic source at the tissue surface via thermo-elastic energy conversion. The generated acoustic wave propagates like a traditional ultrasound source and echoes back to the tissue surface for detection. The returning acoustic waves are measured via optical methods such as laser Doppler vibrometery (LDV). A combination of laser source and receiver converts any tissue surface into a viable ultrasonic transmitter and receiver, enabling full non-contact, large-area, operator-free ultrasonic imaging.

LUS and the photoacoustic effect may be used as means to couple acoustic energy into a human subject. The photoacoustic effect is a well-known process by which optical energy, typically from a laser, is absorbed by a medium. This transfer of energy results in a thermal expansion of the medium, which will result in a propagating acoustic and/or elastic wave. In one configuration, LUS converts optical energy to acoustic energy at the tissue surface, minimizing the effects of optical attenuation in tissue while maximizing acoustic energy output and imaging depth. Many of the properties of the resulting acoustic and/or elastic wave can be controlled by the source laser within the material limitations of the source medium. Using a laser system eliminates coupling gels that are conventionally used in ultrasound imaging and applied to the patient's skin that can contaminate open body tissues. In addition, a laser system can provide fine spatial and temporal resolution to yield higher quality images while reducing distortion observed with contact sensing deformation. Biomedical photoacoustic systems can use laser wavelengths in the visible to near infrared (i.e., 400-1100 nm), which have absorption depths of approximately 0.1-10 cm. However, the actual penetration depth is usually less than the absorption depth due to significant optical scattering. In addition, existing photoacoustic systems utilize a single source of optical illumination with a fairly weak resultant acoustic response, making it difficult to probe structures within the patient.

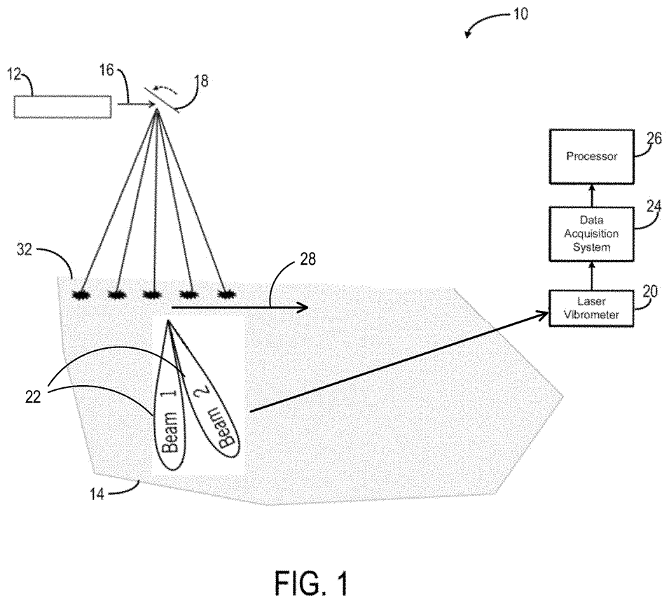

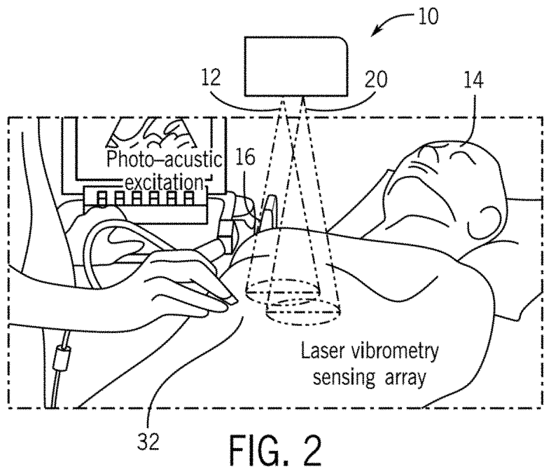

Referring to FIGS. 1 and 2, a system 10 is shown that provides a means to propagate acoustic energy in a single direction so that the acoustic amplitude can be larger than that induced via standard photoacoustic means. The system 10 may include a photoacoustic excitation source 12, which may be a LUS, configured to transmit acoustic disturbances, or acoustic energy, or light, into a patient 14 to induce propagating photoacoustic waves. The photoacoustic excitation source 12 may be, for example, a directed source of radio frequency energy or microwave energy. The photoacoustic excitation source 12 may be coupled to a fixed frame located around a subject. Alternatively, the photoacoustic source 12 may be a handheld device, such as a laser source configured to produce a modulating frequency between about 0 Hz to 10 MHz. Likewise, the photoacoustic source 12 may be a continuous wave (CW) laser. The photoacoustic source 12 may be arranged remotely from the patient 14 and produce a laser beam 16 directed at a scanning mirror 18 to transmit the acoustic energy into the patient 14. A sensor 20, for example, a laser vibrometer sensing array or an ultrasonic transducer receiver, may be positioned remotely from the patient 14 to detect vibrations created by backscatter of a resultant wave 22, as shown in FIG. 1. The resultant wave 22 may be propagating photoacoustic waves generated on or within the patient 14. A data acquisition system 24, as shown in FIG. 1, may be coupled to the sensor 20 to receive the resultant wave 22 and a processor 26 may be coupled to the data acquisition system 24. The processor 26 may be configured to create coherence addition of the transmit/receive signal in post processing, such as during image reconstruction.

As shown in FIG. 1, the processor 26 may be configured to translate the acoustic energy along the patient 14 in a defined direction 28, as indicated by the arrow in FIG. 1, by rotating either the scanning mirror 18 or translating the photoacoustic excitation source 12 such that the laser spot incident upon the patient moves at the speed of sound, for example. This translation results in producing the resultant wave 22. The beam 16 is moved along the defined direction 28. For example, the mirror 18 may be moved to thereby move the beam 16 along the defined direction 28. Also, though more cumbersome, the source 12 can be moved. The processor 26 may also be configured to measure the vibrations at a surface 32 of the patient 14. The vibrations are created by the backscatter of the resultant wave 22 from probing the structures within the patient 14. The processor 26 may then generate ultrasound images of the structures within the patient using the vibrations detected by the sensor 20. Resultant wave 22 may be a wave generated in any direction, such as a wave propagating laterally to the defined direction 28, as indicated by beam 1, or may be a wave propagating at some other angle in the patient 14, as indicated by beam 2.

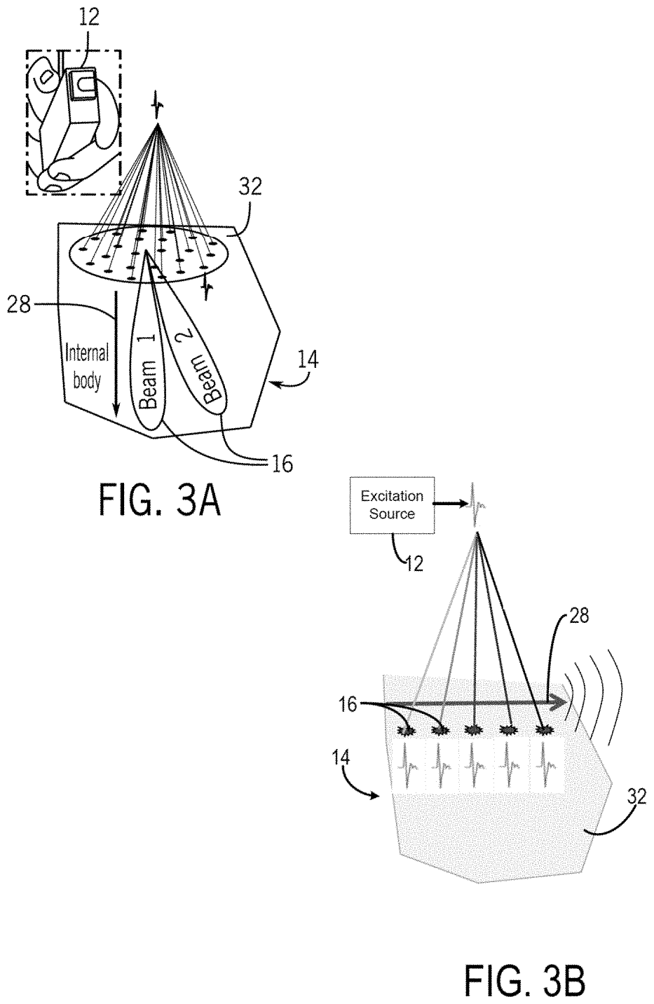

Referring to FIG. 3A, the photoacoustic excitation source 12 is shown as a handheld laser source. The handheld laser source may be used to generate elastic wave propagation into the body. The non-contact laser excitation source generates acoustic/ultrasonic waves that travel into the patient 14 and return back to the skin surface 32. The laser source may be timed/phased to transmit the laser beam 16 that propagates in the defined direction 28 in order to scan the structures of the patient 14 at locations deeper than the optical penetration depth. The laser source may be, for example, a low-powered laser system that can generate acoustic/ultrasonic bandwidths and power levels that propagate into the patient 14, return to the skin surface 32, and that can be readily used to form ultrasound images. Thus, the laser source provides a controlled directional source of acoustic energy to probe and image specific structures of the patient 14. Advantageously, the acoustic and/or elastic waves have a directionality that eases the ability to generate three-dimensional ultrasound images.

Alternatively, as shown in FIG. 3B the photoacoustic excitation source 12 may be translated along the patient 14, for example, at the speed of sound, in a defined direction. The coherent summation of the propagating photoacoustic waves may be performed during post-processing. Coherent summation of the propagating photoacoustic waves has the advantage that the wave amplitudes, rather than intensities, add, leading to a stronger overall resultant wave. The resultant wave may propagate along the defined direction to probe structures of the patient's 14 body.

Again referring to FIGS. 1 and 2, once the photoacoustic excitation source 12 is used to induce the propagating photoacoustic waves into the patient 14, the waves scatter, reflect, and refract in relation to the tissue mechanical property contrasts. As the propagating photoacoustic waves are backscattered towards the surface 32, vibrations are induced at the surface 32. The vibrations may be detected and measured remotely without contacting the patient 14 using the sensor 20 (i.e., the laser vibrometer sensing arrays). The sensor 20 operates as interferometer, for example, that emits a beam of light configured to be safely delivered to an eye or skin of the patient 14. The sensor 20 may be for example, a Coherent Multipixel Imaging system or a Digital Focal Plane Array (DFPA). The sensor 20 receives the spatially distributed acoustic/ultrasonic return from the body interior. These signals are then processed by the processor 26, as shown in FIG. 1, to form structural 2D and 3D ultrasound images of the interior structures of the patient 14.

The sensor 20 can measure vibrations over a frequency band from 1 Hz ranging to 40 MHz, for example. The sensor 20 may include firmware, for example, that utilizes Doppler tracking to compensate for movement of the patient 14. Further, the sensor 20 may provide motion-compensation capabilities that enable measurement of transmitted elastic waves in the body from a moving reference such as the handheld laser source 12, as shown in FIG. 3A. Thus, elastic wave excitation and measurement can be performed from as little as a few inches away from the patient 14 to as much as 30 meters, for example, from the patient 14.

In one non-limiting example, the photoacoustic excitation source 12 may be an optical source configured to generate acoustic and elastic waves in the body of the patient 14 from a standoff—noncontact position. More specifically, the optical source may generate a short optical frequency pulse to initiate and generate ultrasonic waves into tissue of the patient 14, which are driven by the primary mechanism of photoacoustic phenomena. Photoacoustic phenomena first develop from the photons that impinge on a target surface emitted from an optical source and the conversion of the photons into heat by the absorbing material, such as a fluid or biological tissue complex. This process may be a nonlinear thermal shock loading that enables low Q tissue to deform rapidly and thus, generates ultrasonic acoustic and elastic waves.

In a first stage of the photoacoustic process, photons are absorbed by particles comprising a tissue volume, where the absorption coefficient μₐ is described below by equation (1):

$${μ}_{a} = ρ {σ}_{a} where , {σ}_{a} = - 4 \frac{2 π a}{λ} π {a}^{2} Im \{ \frac{{n}_{1} - {n}_{0}}{{n}_{1} + 2 {n}_{0}} \} \qquad ( 1 )$$

where ρ and σₐ are the particle density and cross-sectional area, respectively, and a is the particle radius, where a<<the optical wavelength, and n₁ and n₀ are the refraction indices, respectively, of the absorbing material and an infinite homogeneous non-absorbing medium.

For an optical pulse incident on tissue particles, the total absorbed energy, Eₐ may be described according to equation (2) below:

Eₐ(r,t)=μₐ∫_{4π}I(r,t,ŝ)dΩ=μₐU^{inc}(r,t) (2)

Where I is the specific intensity absorbed by the tissue particles at a position r from light incident in a direction ŝ. U^{inc} may be the average incident intensity with units of J/cm². The average incident intensity may be of particular concern when developing an optical laser ultrasound where the intensity is within eye and skin safe limits for the duration of optical radiation. In one example, 1-20 mJ/cm² is likely to meet safety requirements in the operational system 10.

The governing relationship establishing tissue deformation and thus, acoustic or elastic wave generation evolves from the tissue temperature increase caused by the absorbed energy as shown in equation (3) below:

$${ρ}_{m} C \frac{\partial T ( r , t )}{\partial t} - κ {∇}^{2} T ( r , t ) = {E}_{a} ( r , t ) \qquad ( 3 )$$

Where ρₘ,C,κ,T are the tissue mass density, specific heat, thermal conductivity, and temperature, respectively. The first term shown in equation (3) describes the temperature increase due to optical absorption and diffusion. The optical diffusion may be several orders of magnitude larger than that of the thermal diffusion, thus, the second term shown in equation (3) may be negligible and the temperature increase due to the optical pulse radiation can be described by equation (4) below:

$$\frac{\partial T ( r , t )}{\partial t} \approx \frac{1}{{ρ}_{m} C} {μ}_{a} {U}^{i n c} ( r , t ) \qquad ( 4 )$$

In addition, equation (4) may imply that thermal diffusion can be neglected since the optical pulse duration is considerably smaller than the time scale of thermal diffusion.

The effect of optical propagation into a scattering media, such as complex biological tissues, may be another component to understanding the process of photoacoustic phenomenology. Typically, the materials comprising tissue mass are considerably heterogeneous, where blood hemoglobin, for example, is highly absorptive to light while other tissue cells are simultaneously, highly reflective. Light and optical frequency waves may propagate in tissue and can be described by a diffusion approximation as shown in equation (5) below. The diffusion of the optical average intensity, U due to an incident energy density, S₀ is as follows:

$$D {∇}^{2} U ( r , t ) - \frac{1}{c} \frac{\partial U ( r , t )}{\partial t} - \overline{{μ}_{a}} ( r ) U ( r , t ) = - {S}_{0} ( r , t ) \qquad ( 5 )$$

In equation (5) above, D may be the optical diffusion coefficient and c may be the average speed of light in the tissue. The average intensity experienced in a homogeneous scattering tissue column can then be related to the average incident energy as a function of frequency according to equation (6) below:

$$\tilde{U} ( r , ω ) = {\tilde{U}}^{i n c} ( {r}_{s} , r ) + \frac{1}{4 π} {\int }_{V} {\tilde{U}}^{i n c} ( {r}_{s} , {r}^{′} ) Δ {μ}_{a} ( {r}^{′} ) \times g ( {γ}_{0} \vert r - {r}^{′} \vert ) d {r}^{′} \qquad ( 6 )$$

In equation (6) above, g may be a 3D Green's function, for example, and γ₀ may be the frequency-dependent wave number for the optical diffuse photon density wave. The average incident energy can be derived showing the relationship between the incident energy density in the time domain according to equation (7) below:

$${U}^{i n c} ( {r}_{s} , r , t ) = \frac{{s}_{0}}{{( 4 π D c t )}^{3 / 2}} exp [ \frac{\vert {r}_{s} - r \vert}{4 D c t} - {μ}_{a} \vert {r}_{s} - r \vert ] \qquad ( 7 )$$

The acoustic or elastic wave that can be measured by the sensor 20, such as an optical receiver including a laser Doppler vibrometer or conventional contact transducer, is another component to describing photoacoustic conversion of light to pressure and resultant acoustic wave propagation. For simplicity, an inviscid fluid may be used to demonstrate the generation and propagation of the longitudinal or compressional wave from incident light, as shown in the linear force equation (8) below:

$${ρ}_{m} \frac{{\partial }^{2} u ( r , t )}{\partial {t}^{2}} = - ∇ p ( r , t ) \qquad ( 8 )$$

where u may be the acoustic displacement and p may be the acoustic pressure. The tissue media may then deform from expansion according to equation (9) below:

$$∇ \cdot u ( r , t ) = - \frac{p ( r , t )}{{ρ}_{m} {v}_{s}^{2}} + β T ( r , t ) \qquad ( 9 )$$

where β is the volume expansion coefficient and vₛ is the acoustic speed in the tissue.

$${∇}^{2} p ( r , t ) - \frac{1}{{v}_{s}^{2}} \frac{{\partial }^{2} p ( r , t )}{\partial {t}^{2}} = {ρ}_{m} β \frac{{\partial }^{2} T ( r , t )}{\partial {t}^{2}} \qquad ( 10 )$$

Combing equations (9) and (10) above, the relationship between the heat source and the resultant pressure is shown below in equation (11) in terms of the optical average intensity and optical absorption coefficient:

$${∇}^{2} p ( r , t ) - \frac{1}{{v}_{s}^{2}} \frac{{\partial }^{2} p ( r , t )}{\partial {t}^{2}} = \frac{β}{C} [ {μ}_{a} + Δ {μ}_{a} ( r ) ] \frac{\partial U ( r , t )}{\partial t} \qquad ( 11 )$$

The pressure distribution along the tissue column resolves to equation (12):

$$p ( r , t ) = {p}_{0} ( r , t ) + \frac{β}{4 π C} {\int }_{V} \frac{d {r}^{′}}{\vert r - {r}^{′} \vert} Δ {μ}_{a} ( {r}^{′} ) \times {\vert \frac{\begin{matrix}\partial {U}^{i n c} \\ ( {r}^{′} , {t}^{′} )\end{matrix}}{\partial {t}^{′}} \vert}_{{t}^{′} = t \frac{\vert r - {r}^{′} \vert}{{v}_{s}}} \\ Where \\ {p}_{0} ( r , t ) = \frac{β {μ}_{a}}{4 π C} {\int }_{V} \frac{d {r}^{′}}{\vert r - {r}^{′} \vert} {\vert \frac{\partial {U}^{i n c} ( {r}^{′} , {t}^{′} )}{\partial {t}^{′}} \vert}_{{t}^{′} = t - \vert r - {r}^{′} \vert / {v}_{s}} \qquad ( 12 )$$

In equation (12) above, p₀(r,t) may be the incident pressure at the onset of the tissue column.

Once the photoacoustic excitation source 12 described above transmits acoustic disturbances into the patient 14, the sensor 20, such as a noncontact laser vibrometer sensing array, may measure the ultrasonic returns. The ultrasonic returns may be stimulated by the optical excitation sources that arrive from internal boundaries composing structures and material property distributions inside the patient 14, for example. In one non-limiting example, the sensor 20 is an optical heterodyne ladar design utilized for the vibrometer sensing system.

In conventional heterodyne detection, a signal of interest at a known frequency is non-linearly mixed with a reference “local oscillator” (LO) that is set at a close-by frequency. The desired outcome may be the difference frequency, which carries the signal information (i.e., amplitude, phase, and frequency modulation) of the original higher frequency signal, but is oscillating at a lower more easily processed carrier frequency. Electrical field oscillations in the optical frequency range cannot be directly measured since the relatively high optical frequencies have faster oscillating fields than electronics can respond. Instead, optical photons are detected by energy or equivalently by photon counting, which are proportional to the square of the electric field and thereby form a non-linear event. Thus, when the LO and the signal beams impinge together on a target surface, such as the surface 32 of the patient 14, the LO and signal beams “mix” and produce heterodyne beat frequencies.

The performance of a laser vibrometer, for example, and the process of ultrasonic wave measurement may be determined by the noise floor of the laser vibrometer. The noise floor may include, but is not limited to, 1) shot noise that dominates the noise floor at ultrasonic frequencies, 2) speckle noise that contributes noise in the audible acoustic band, and 3) platform and subject target vibration caused by motion by a variety of potential sources other than the intended system optical excitation source.

Shot noise may arise from statistical fluctuations in measurements. The detected electrical current for a heterodyne ladar may be described according to equation (13) below:

i(t)=l_{LO}iₛ(t)+2ηₕi_{LO}iₛ(t)cos [ω_{IF}t+θ(t)] (13)

where i_{LO} and iₛ(t) are the currents from the local oscillator and signal, ηₕ is the heterodyne mixing efficiency (0 to 1), ω_{IF} is the intermediate frequency (carrier signal is mixed with the local oscillator to produce a difference or beat frequency to improve signal gain), and θ(t) is the phase shift. ω_{IF} is equal to the acousto-optic modulator frequency offset plus the Doppler offset due to platform motion. Thus, the phase shift may be described according to equation (14) below:

$$θ ( t ) = 2 k x ( t ) + {θ}_{s} ( t ) = \frac{4 π x ( t )}{λ} + {θ}_{s} ( t ) \qquad ( 14 )$$

where x(t) is the line-of-sight distance between the heterodyne ladar and tissue surface 32, θₛ(t) is the random phase of the speckle lobe, and is the optical wavelength of the laser vibrometer. x(t) may change due to body vibrations and movement, laser platform vibration, and pointing jitter, for example.

The laser vibrometer sensing arrays 20 may be characterized by the carrier-to-noise ratio (CNR). More specifically, the received number of photoelectrons per second, ϕₑ (i.e., optical return from the vibrating tissue surface) over the vibrometer demodulated bandwidth may determine the received signal quality. The greater the number of photoelectrons received by the laser sensing system, for example, the lower the shot noise is, thus, resulting in a more sensitive laser vibrometer 20. In some embodiments, the CNR may be increased by increasing the power of the laser vibrometer 20 and by decreasing the laser beam 16 diameter that impinges upon the tissue surface 32.

The shot noise spectrum of the surface particle velocity, A_{v,sh} as a function of frequency, f, may be proportional to the received returning photoelectrons as described by equation (15) below:

$${A}_{v , sh} ( f ) = \frac{f λ}{\sqrt{{φ}_{e}}} \qquad ( 15 )$$

As previously described, another source of noise may be from speckle, for example. Speckle is the noise that occurs due to the distribution of optical scatters on the tissue surface 32 encountered by the laser beam 16. For a diffuse surface, for example, there may be many scatterers (based on surface roughness) that reflect light back to the receiver. The speckle noise contribution to the laser vibrometer 20 can be reduced by signal time integration with respect to the same realization of scatterers. Increasing the integration time may reduce speckle noise and thus improve the sensitivity of the system 10. However, if during the allotted integration summing time, the laser beam 16 changes position on the target surface 32 due to platform motion, beam jitter, or target movement, for example, the speckle realization may change thereby creating translation or dynamic speckle and increase in the noise floor. Faster laser beam 16 translation speeds across the surface 32 of the patient 14 may also increase the speckle noise floor contribution. The speckle noise amplitude may be described according to equation (16) below:

$${A}_{v , sp} ( f ) = λ \sqrt{\frac{π {f}_{e x c}^{2}}{12}} \sqrt{\frac{2 α}{{α}^{2} + {( 2 π f )}^{2}}} \qquad ( 16 )$$

where α=2πf_{exc} and f_{exc}=vₜ/d (i.e., laser beam translation velocity on target over the laser beam diameter) is the exchange rate of the speckle pattern.

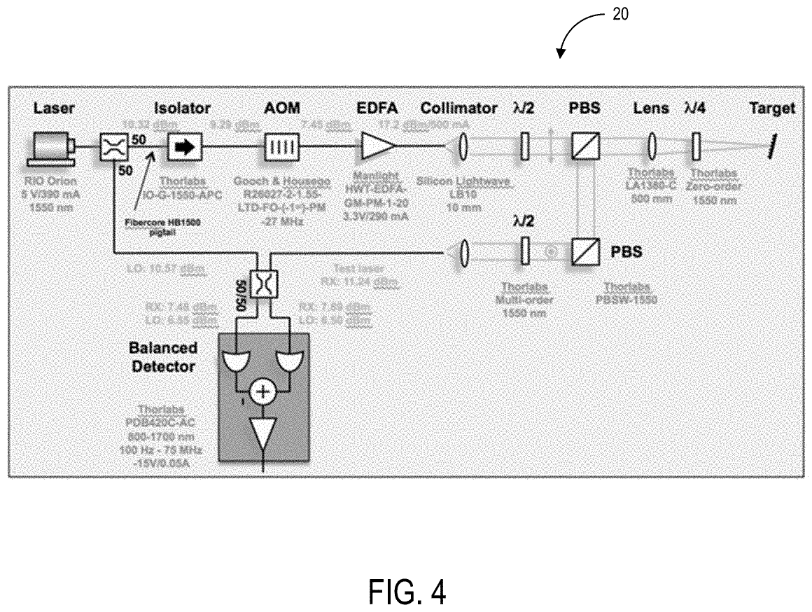

Referring to FIG. 4, a non-limiting example laser vibrometer 20 is shown. Performance of the laser vibrometer, such as the laser Doppler vibrometer 20 shown in FIG. 4, for ultrasonic measurements may be characterized by the shot noise contribution (e.g., at 1 MHz), for example, that is anticipated to dominate the noise floor sensitivity. However, when introducing system motion, speckle noise may become a significant factor. Even subtle motion with a small laser beam 16 diameter (on the order of millimeters) can produce significant fluctuations in the speckle realization and resultant noise floor.

As described above, the photoacoustic, or LUS, source laser delivers packets of optical energy to the target surface. The target then absorbs the optical energy causing rapid heating and the induced mechanical stress from the rapid heating then propagates into the tissue as an ultrasonic wave. Reflected waves from various internal boundaries in the target can be measured at the target surface using a laser vibrometer. Since the source and receive lasers generate and measure on the surface of the target, respectively, the spatial location of the source and receiver may be characterized to accurately reconstruct an image.

Point Tracking and Surface Profile Characterization

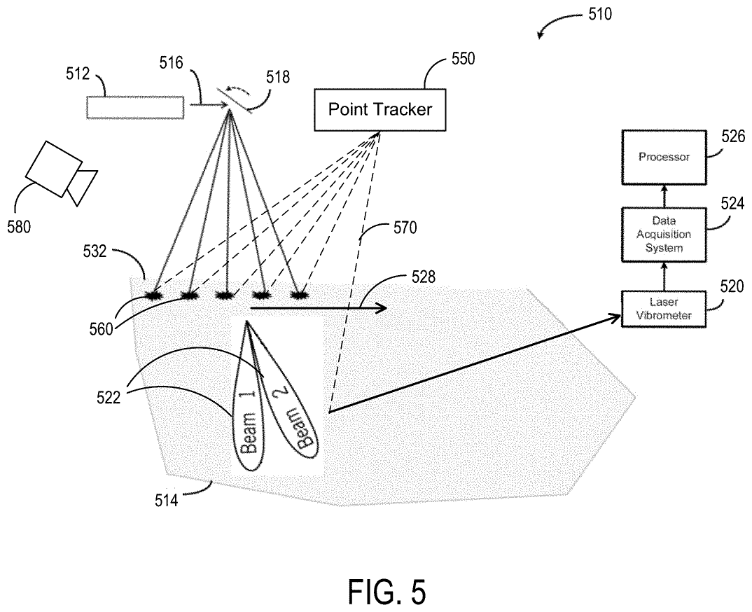

Referring to FIG. 5, a system 510 is shown that provides a means to propagate acoustic energy in a subject 514 and to perform image reconstruction, such as for 2D or 3D image reconstruction of the surface of the subject or of an internal structure of the subject. The system 510 may include a photoacoustic excitation source 512, which may be a LUS, configured to transmit acoustic energy into a subject 514 to induce propagating photoacoustic waves. As described above, the photoacoustic excitation source 512 may be, for example, a directed source of radio frequency energy or microwave energy or a handheld device, such as a laser source. The photoacoustic source 512 may be a continuous wave (CW) laser, or a pulsed laser. The photoacoustic source 512 may be arranged remotely from the subject 514 and produce a laser beam 516 directed at an optional scanning mirror 518, or moved by a linear stage, to transmit the acoustic energy into the subject 514. In some configurations, photoacoustic source may include a plurality of laser beams 516. A sensor 520, such as a laser vibrometer sensing array, an interferometer, or an ultrasonic transducer receiver, and the like, may be positioned remotely from the subject 514 to detect vibrations created by backscatter of a resultant wave 522. In some configurations, sensor 520 may be a pulsed laser. Coherent summation of the propagating photoacoustic waves may be performed in post-processing. A data acquisition system 524, may be coupled to the sensor 520 to receive the resultant wave 522 and a processor 526 may be coupled to the data acquisition system 524.

In some configurations for image reconstruction, point tracking system 550 may be used. In some applications, such as human imaging, the skin surface is neither flat nor completely static. In a clinical setting, use of LUS for human applications may be optimized with implementation of additional systems and/or methods to adapt LUS for human skin characteristics. Since the spatial location of the source spot 560 and detection or receiver spot 570 may be dependent on where the transmit and receive laser land on the skin surface 532, tracking of the transmit and receive points using the point tracker 550, and/or prior knowledge of the skin surface 532 geometry, may be used for accurate image reconstruction. Whereas traditional ultrasound image reconstruction may only rely on prior knowledge of the spatial location of each piezoelectric transmitter and receiver in the ultrasound probes, in accordance with the present disclosure a photoacoustic system, or LUS system, may be implemented with tracking of each transmit and receive location in free space.

In some configurations, the relative location of the source spot 560 and receiver spot 570 locations may be used. In a portable or hand-held configuration, a shared coordinate system between the spot locations may be used. Such a shared coordinate system may provide for correcting for moving reference frames between the hand-held device and the subject. In some configurations of a portable system, accelerometers may be used in the hand-held device to provide for a shared coordinate system or for tracking relative locations between a source and a receiver. In a fixed-frame or non-portable configuration, such as a table top apparatus, accelerometers and the like may not be used and a shared coordinate system may be provided by the known positions of the sources on the fixed-frame.

In one configuration, photoacoustic, or LUS, source spot 560 and receiver spot 570 locations on the surface 532 may be tracked during scanning using point tracker 550 to enable robust ultrasound image reconstruction of the interior of non-flat objects, non-uniform sampling techniques, and through transmission imaging techniques, and the like. 2D and/or 3D spatial locations of the source spot 560 and receiver spots 570 acquired with the point tracker 550 may be used to provide a spatial reference location for ultrasound image reconstruction. Point tracker 550 may include, but is not limited to: stereo, structured light, or optical cameras, LIDAR, time of flight measurements, calibration locations on the surface 532, 3D surface characterization of the target prior to scanning, and the like. Tracking techniques may provide the spatial location of the source spot 560 and receiver spots 570 for image reconstruction. Source spots 560 and receiver spots 570 may also be actively tracked using point tracker 550 during the scan for real-time image reconstruction.

In some configurations, active subject motion tracking and compensation using a separate camera 580 may be used for real-time patient skin surface 532 tracking. Adaptive focusing of either source or detection lasers may use active feedback of the skin surface 532 for accurate tracking. In some configurations, the location of the photoacoustic source 512 and the sensor 520 are tracked in order to enable ultrasound 2D and/or 3D image reconstruction. In some configurations, a spot location and motion may be determined by automatically detecting, computing, and tracking skin features. Each time an image is acquired, the system may synchronously record a skin image and identifies skin features; feature descriptors may be automatically computed. Skin features result from uneven distribution of melanin and hemoglobin pigments, pores, and skin surface texture and allow to uniquely identify locations on the skin. Using the recovered positions and motion, a three-dimensional body map may be constructed. Each image may be accurately localized with respect to the map—a process of simultaneous localization and mapping.

The processor 526 may be configured to translate the acoustic energy, or beam 516, along the subject 514 in a defined direction 528, as indicated by the arrow, by rotating either the scanning mirror 518 or translating the photoacoustic excitation source 512. The processor 526 may also be configured to create coherence addition of the transmit/receive signal in post processing, such as during image reconstruction by superposition of the detected waves from each transmit point, or by the use of multiple transmitter/receivers operating to amplify the transmit power or receive sensitivity, respectively. The processor 526 may also be configured to measure the vibrations at a surface 532 of the subject 514. The vibrations are created by the backscatter of the resultant wave 522 from probing the structures within the subject 514. The processor 526 may then generate ultrasound images of the structures within the patient using the vibrations detected by the sensor 520.

Once the photoacoustic excitation source 512 is used to induce the propagating photoacoustic waves into the subject 514, the waves scatter, reflect, and refract in relation to the tissue mechanical property contrasts. As the propagating photoacoustic waves are backscattered towards the surface 532, vibrations are induced at the surface 532. The vibrations may be detected and measured remotely without contacting the subject 514 using the sensor 20 (i.e., the laser vibrometer sensing arrays). The sensor 520 operates as interferometer, for example, that emits a beam of light configured to be safely delivered to an eye or skin of the subject 514. The sensor 520 may be for example, a Coherent Multipixel Imaging system or a Digital Focal Plane Array (DFPA). The sensor 520 receives the spatially distributed acoustic/ultrasonic return from the body interior. These signals are then processed by the processor 526 to form structural 2D and 3D ultrasound images of the interior structures of the subject 514.

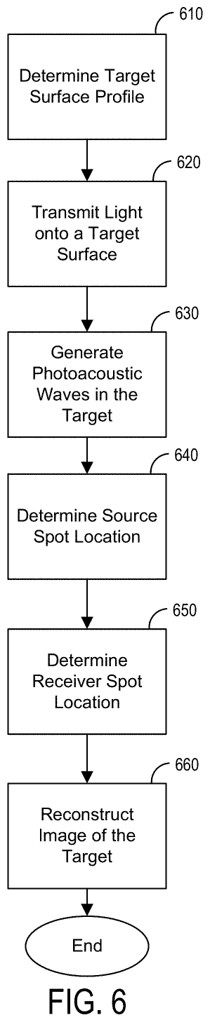

Referring to FIG. 6, non-limiting example steps for performing image reconstruction of a subject using the system of FIG. 5 are shown. A target surface profile may be determined at step 610. The surface may be a 3D surface. The surface profile may be determined from ultrasound measurements made prior to photoacoustic excitation of the subject, or may be made by a separate camera system as described above, and the like. Light, such as laser light, may be transmitted onto a target surface at step 620. Photoacoustic waves may be generated in the subject target at step 630. Source spot location, such as an incident laser light point or spot, may be tracked or determined at step 640. Receiver spot location, such as a vibrometer laser light measurement point or spot, may be tracked or determined at step 650. An image of the target using the data of the target surface profile from step 610, or the source spot location from step 640, or the receiver spot location from step 650, or any combination thereof, may be reconstructed at step 660. In a non-limiting example, the image may be a 3D image, if the data is 3D data.

Laser Adjustment

In some configurations, additional transmit and receive lasers, or a plurality of sources and/or detectors, may be used for photoacoustic imaging at clinically relevant time scales and quality. Traditional ultrasound has hundreds of piezoelectric elements and can be simultaneously used to focus acoustic energy at specific spatial locations to amplify acoustic energy and signal sensitivity. In one configuration, a multi-element focusing method is provided that includes tracking the surface geometry and setting the appropriate time delay parameters on the laser transmitter to focus the generated acoustic wave within the tissue. Implementing acoustic focusing using lasers can increase total acoustic energy output and increase image quality and acquisition speed.

In one configuration, a pulsed laser source may be used and a continuous wave (CW) detector may be used. High-energy, fast-repetition lasers may improve signal to noise ratio and reduce data acquisition time for any optical-based ultrasound technology. Parallelization of optical sources and detectors may further improve photoacoustic and LUS technology by creating optical ultrasound arrays for transmission or detection

The pulsed laser source and CW detector may use an appropriate level of power to be safe for human applications. In some configurations, the laser source may operate in a wavelength range of 1500-1800 nm. In a non-limiting example, the laser source may be selected to operate at a wavelength of 1540 nm, which may be selected to maximize the photoacoustic source amplitude by leveraging the high optical absorption of biological tissue.

In one configuration, a source wavelength may be selected to take into account patient safety and system design. Since optical to acoustic conversion is a thermal effect, spectral regions inducing thermal or thermomechanical processes in tissue may be desired. A user may select or tune a source to correspond to peaks in the optical absorption coefficient in biological tissue such that the optical source can both limit optical penetration into the tissue while maximizing the thermoelastic conversion efficiency. In biological tissue, light induced thermal effects are maximized in the infrared (IR) spectrum due to the absorption characteristics of water in the tissue.

In one configuration, a sensor or detector may be selected to take into account system complexity and patient safety with a desire to maximize reflected light. Interferometric detection of acoustic waves may be bounded by the absolute quantity of returning light from the tissue surface rather than the quantity of light absorbed in the tissue. Absolute quantity of returning light may be dictated by the tissue reflectance characteristics and the irradiance of the interferometric light source. In some configurations, retroreflective materials can be used to boosts optical reflectance of the target surface and increase detection sensitivity. For biological tissue, reflectance may be wavelength dependent and permissible irradiance is subject to allowable limits. In some configurations, assuming similar photodetector responsivity across the spectrum, wavelength selection may be made to maximize reflected light while remaining within the limits for safety. Tissue reflectance is approximately the inverse of the tissue absorbance.

In one non-limiting example, the sensor or detector may be selected to operate between 1500-1800 nm, with acoustic time signals around hundreds of microseconds. For applications where the eye of a subject may be involved, disregarding eye safety may require providing eye protection to a subject, complete enclosure, or specialized clinical facilities. Wavelengths in the range disclosed in the present disclosure may maximize reflected light from the tissue while balancing system complexity and patient safety.

In some configurations, soft tissue and/or bone features may be detected using a system according to the present disclosure. Tissue features such as tendons, muscle boundaries, bone surfaces, and the like may be detected using appropriate source parameters as discussed above. In a non-limiting example of imaging a forearm, subcutaneous fat layer may be seen in the top 0.5 cm of reconstructed images, the tendon from 0.5 cm to 1 cm, and the surface of the ulna, or other bone, from 2 cm to 2.5 cm.

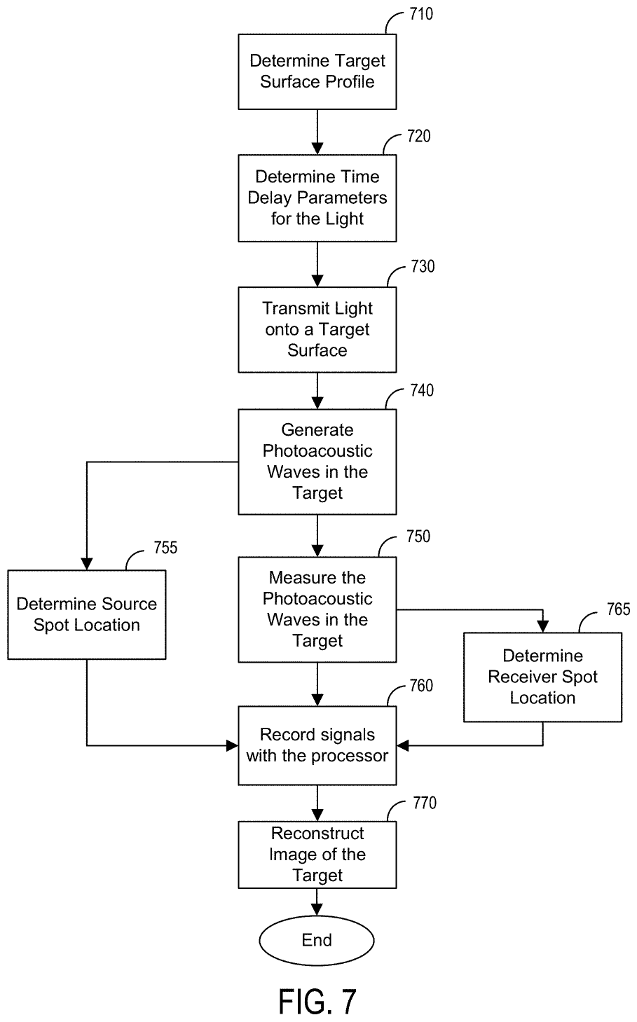

Referring to FIG. 7, non-limiting example steps for performing image reconstruction of a subject using time delay from a plurality of sources are shown. A target surface profile may be determined at step 710. The target surface profile may be acquired in 2D and/or 3D, and the like. As described above, the target surface profile may be determined based upon ultrasound measurements, optical measurements, and the like. Time delay parameters for transmitted light may be determined at step 720 and these parameters may be used when the light is transmitted to the target surface at step 730. The transmitted light with the delay parameters may be used to optimize photoacoustic wave generation in the target at step 740. The source spot locations may optionally be tracked as described above at step 755. The photoacoustic waves generated in the target may be measured at step 750, such as by use of a sensor or detector as described above. The receiver spot locations may optionally be tracked as described above at step 765. In some configurations, either the source spot locations may be determined as step 755, or the receiver spot locations may be determined as at step 765, but may not both be determined. In other configurations, both source spot and receiver spot locations are determined with both steps 755 and 765 being used. The measured signals may be recorded with the processor at step 760. An image, such as a 2D or 3D image of the target, may be reconstructed at step 770.

In some configurations, an amplitude modulated source laser or a pulsed detection lasers may be used. Implementing a fast amplitude modulated optical source may improve imaging depth, bandwidth, and resolution. Acoustic signal-to-noise ratio may also be amplified with techniques such as pulse compression, matched filtering, source encoding, and the like. Previously, conventional LUS systems have used continuous wave detection lasers. Pulsed detection lasers, when used in accordance with the present disclosure, may be advantageous to reduce overall optical exposure. Eye and skin safety may limit the allowable average optical power on the surface. Total measurement time for each transmit may be short, meaning significant portions of the optical energy delivered onto the skin may not be used for detection. In one non-limiting example, a pulsed laser is only active for the duration of a ˜200 us measurement window, and thereby limits total optical skin exposure and allows for higher peak optical power during measurement. In some pulsed detection laser configurations, optical sensitivity may also be increased along with signal to noise ratio, in addition to improving patient safely. In some configurations, specific acoustic frequency bands may be amplified by time modulating the optical source amplitude, or the photoacoustic source, at the desired acoustic frequency. This may be performed in order to overcome bandwidth limitations when used in biological tissue.

Surface Enhancements

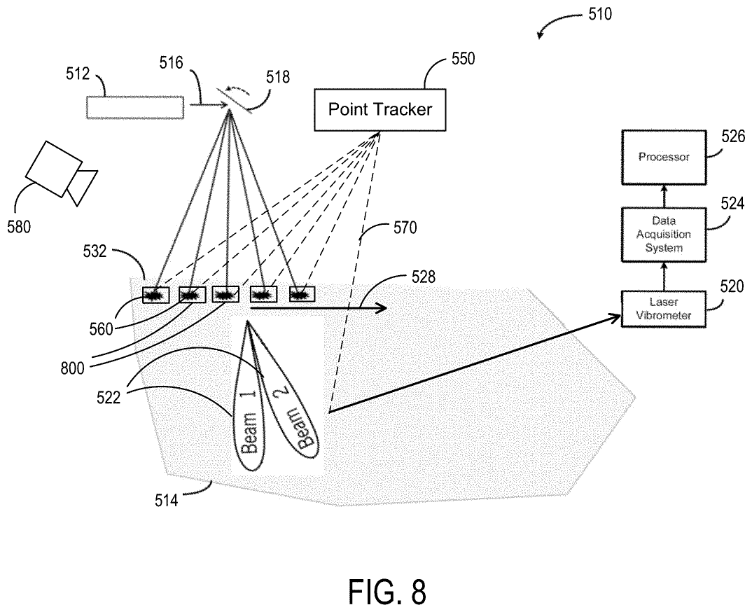

Referring to FIG. 8, the system 510 from FIG. 5 is shown with surface enhancements 800. In some configurations, surface enhancements 800 may be used to improve optical characteristics for the optical to acoustic generation or optical detection. Current LUS methods may be limited by the optical and acoustic properties of the target surface. The target, such as skin tissue, dictates the laser to acoustic conversion efficiency, safe laser to skin exposure limits, and the generated acoustic wave frequency. These characteristics can be controlled by using some surface treatment of the target surface before imaging. In some configurations, the use of gels (such as hydrogels), gel pads, liquids, and the like may be used to optimize surface characteristics for imaging. Materials may also be designed specifically for photoacoustic or LUS imaging to behave in a similar manner to how ultrasound uses gel to couple the piezoelectric transducer to the skin surface. Gel pads placed on the skin surface can enhances the optical to acoustic energy conversion efficiency while bypassing the skin safety limit by protecting the skin surface from direct laser irradiance. A surface enhancement layer could expand the source bandwidth, improve optical detection sensitivity with higher surface reflectance, and permit higher optical exposure limits, among other benefits. In some configurations, optical reflectors may be embedded in the gel pad to enhance optical scattering characteristics to increase reflected light for better optical detection. Such optical reflectors may include glass beads, retro reflective dust particles, and the like. In some configurations, the use of gels, gel pads, liquids, or other material designed to improve surface characteristics may be used.

In a non-limiting example, the surface enhancement may be a hydrogel. The water in hydrogels rapidly absorb infrared light and generate a photoacoustic source within the thickness of the hydrogel. Hydrogel uses in biomedical applications include biosensors, artificial tissue, and drug delivery vehicles. A hydrogel surface enhancements layer placed on the tissue surface may provide a way to increase and control the LUS source frequency by utilizing the high water content of hydrogels and fine-tuning of the hydrogel structure, thickness, and mechanical characteristics. The hydrogel surface texture, optical absorption, and reflectivity properties may be adjusted for controlling LUS generation and detection performance. Hydrogel optical absorption may also influence the center frequency of the generated US wave. The surface roughness of the hydrogels may influence the frequency bandwidth of the generated wave, where lower surface roughness may generate a broader frequency LUS source. The water content, thickness, surface roughness of the hydrogel may all be optimized to enable generation of a desired US frequency. The surface roughness and hydrogel reflectivity may control the laser detection of sound.

In a non-limiting example, a hydrogel enhancement layer may be a polyacrylamide hydrogel. The hydrogel may be selected for high fracture toughness and physical flexibility to conform and attach to a surface, such as a skin surface. Hydrogels over a range of water-content may be selected for controlling center frequencies of generated sound, and surface textures for controlling bandwidth of generated sound and ability to detect sound. The hydrogel selected may be matched with the laser to the desired imaging frequency. Lower frequency sound propagates deeper into the body, but may come with lower spatial resolution. The beam shape of individual laser spots may control the lateral and elevation focus of the equivalent aggregate array produced by scanning the source and detector laser over the surface of the body. The optical wavelength of an interferometer may also be altered to penetrate the hydrogel and retroreflective particles may be introduced in the hydrogel to boost optical reflectivity.

In one configuration, selecting surface enhancements 800 that may be appropriate for a subject 514 may be based upon optical absorption properties. In a non-limiting example, the optical absorption of water may be considered such that a surface enhancement 800 is selected to contain a high content of water. The selection of surface enhancements 800 may also be based upon a desired level of patient safety, such as selecting a highly optically absorbing material with higher powered sources 512 in order to maintain skin surface safety for the subject 514.

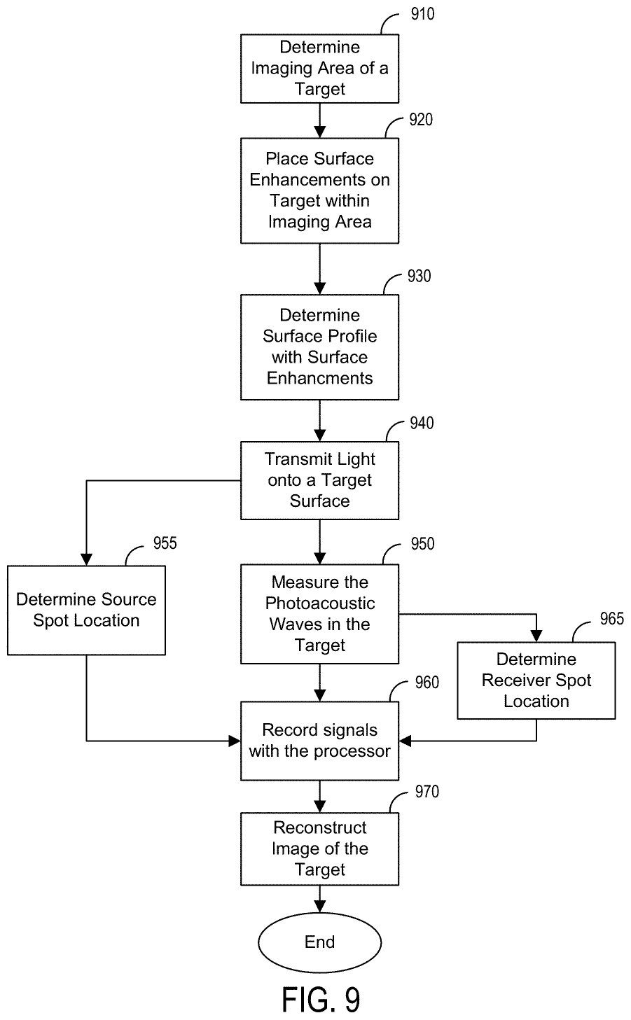

Referring to FIG. 9, non-limiting example steps for performing image reconstruction of a subject using surface enhancements are shown. The location for an imaging area where a surface enhancement may be placed, such as a gel, gel pad, liquid, and the like, may be determined at step 910. The surface enhancement may then be placed on the target subject at step 920. A target surface profile may be determined at step 930 that includes the surface enhancements. The profile may be a 2D profile, a 3D profile, or other profile as desired by a user. Light may be transmitted onto a target surface at step 940. The source spot locations may optionally be tracked as described above at step 955. Photoacoustic waves may be generated in the target and the waves may be measured at step 950, such as by use of a sensor or detector as described above. The receiver spot locations may optionally be tracked as described above at step 965. In some configurations, either the source spot locations may be determined as step 955, or the receiver spot locations may be determined as at step 965, but may not both be determined. In other configurations, both source spot and receiver spot locations are determined with both steps 955 and 965 being used. The measured signals may be recorded with the processor at step 960. An image, such as a 2D image or a 3D image, may be reconstructed of the target at step 970.

Beam Shaping

The shape of a LUS beam, or light, shape on the surface of a subject may be modulated to the surface geometry of a subject or target to optimize the resulting photoacoustic signal for detection. Any shape may be used, such as a circle, line, grid, and the like, determined to optimize optical absorption of the surface of the subject based upon the surface geometry.

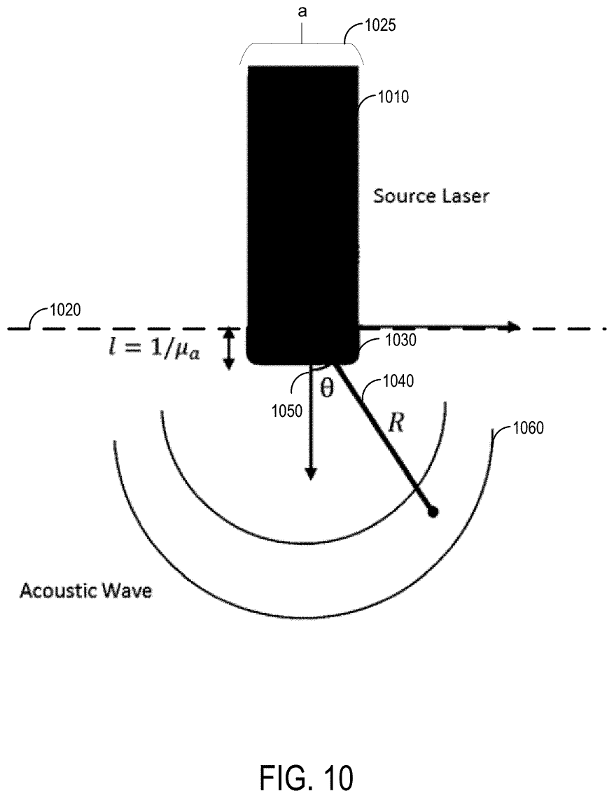

Referring to FIG. 10, a non-limiting example cross section is shown for a source laser beam 1010 incident upon a tissue surface 1020. Beam parameters may include intensity, wavelength, beam diameter “a” 1025, and the like. Penetration depth “1” 1030 may be determined by the tissue optical absorption for the specific optical wavelength used. Penetration depth 1030 may dictate the wavelength of the converted acoustic wave. Geometry of the region around penetration depth 1030 may dictate the parameters of the converted acoustic wave in the tissue. Radius “R” to target 1040 may be at an angle 1050 represented by θ with propagating acoustic waves 1060. Size, geometry, optical power distribution of the optical beam, and the like may determine the converted wave geometry. In a non-limiting example, diameter 1025 of a circular spot may generate an acoustic wave similar to a disk piezoelectric transducer. In non-limiting examples, an optical line, multiple spots, or varying optical power across the beam cross-section can generate various acoustic waves.

Beam Design Parameters May be of the Form:

$${P}_{s} ( ω ) = \frac{i {U}_{0} β {a}^{2}}{2 {C}_{p}} \frac{{e}^{ikR}}{R} \frac{{μ}_{a} k cos θ}{{μ}_{a}^{2} + {k}^{2} {cos}^{2} θ} {e}^{- \frac{{a}^{2} {k}^{2} sin θ}{4}} \qquad ( 17 )$$

Where optical parameters of U₀ represents optical intensity, “a” represents spot radius, and A represents optical wave length. Tissue parameters of β represents coefficient of thermal expansion, Cₚ represents heat capacity. Interaction parameters μₐ(λ) represents optical absorption coefficient. The conversion efficiency may be determined by the cos(θ) fraction immediately preceding the exponential.

In some configurations, to control the converted acoustic wave, specific control of the optical wavelength, energy, and geometry of the irradiating light may be performed. In some configurations, a surface treatment layer may be used to enable control of the surface parameters and remove limitations of the target surface. Source corrections may also be performed to adjust for surface roughness, skin variations, source coherency degradation, and the like.

Influence of surface roughness on resultant pressure wave may be determined by:

$${P}_{r} ( ω ) = {P}_{s} ( ω ) {e}^{- \frac{{σ}^{2} {k}^{2} {cos}^{2} θ}{2}} \qquad ( 18 )$$

Acoustic Attenuation may be determined by:

$$A ( z , ω ) = {A}_{0} 1 {0}^{\frac{a z ω}{40 π}} \qquad ( 19 )$$

Transmit and receive beam parameters may be determined by the properties of the target surface, geometry, and the like. In a non-limiting example, time delays may be computed based on the surface geometry and the desired focusing depth inside the target. Amplitude, wavelength, size, position, shape, time delay and the like of transmit and receive beams may be adjusted based on the surface reflectivity and relative orientation between the source and detectors.

Surface reflectivity may form the basis of a correction as optical detector performance may be determined by the quantity of light returning from a surface, such as a skin surface. Large variations in optical reflectivity may be observed when measuring reflections from skin surfaces. In a non-limiting example, a detailed characterization and model of skin reflectivity may be used to define the limits of LUS on untreated skin, modeling the full LUS imaging process, designing better detectors, and determining LUS use scenarios. The performance of skin-surface optical interferometry may be dependent on several parameters that impact skin reflectivity. Skin reflectivity is a function of variations in surface geometry and constitutive-materials reflective characteristics; the angle of incidence between skin normal and optical path; and relative motion of the skin.

Variations in the skin micro-reliefs may influence directional optical reflectivity in the IR spectrum. These variations in skin micro-relief may be segmented from camera images (such as visible, NIR spectrum cameras, and the like) with the optical reflectivity measurements to correlate how skin variations influence reflectivity in IR. Skin reflectivity may change with angularly resolved reflectivity, physical skin variations, and may be wavelength dependent. For a LUS system, detector sensitivity may be configured to accommodate expected variation with respect to skin viewing angle relative to skin surface normal direction. Skin surface properties may be related to sound detection sensitivity. Micro-relief images may be used in conjunction with angular resolved specular energy reflection measurements in order to relate skin geometry and properties to optical reflectivity as a function of observation angle.

In a non-limiting example, skin surface texture and geometry may be measured using high resolution optical imaging. The angle-resolved skin reflectance versus optical wavelength may also be measured with an orientation-controlled imaging system. Reflected energy of the skin may be measured with a multi-pixel IR sensitive camera. The camera may be mechanically calibrated and aligned with a collimated optical source. In some configurations, the optical source may be tuned, or filtered, to a narrow band of optical wavelengths and may be skin safe for human subjects. The orientation of the multi-pixel IR sensitive camera may be varied with respect to skin surface for a fixed orientation of the source images may be acquired from the camera at defined angular intervals, such as one to two degrees. The average received energy may be determined at each angle by averaging over all pixels. The bulk constitutive-materials reflection characteristics may be determined by averaging over all angles and all pixels at each angle. In some configurations, the steps may be repeated for narrow optical bands, uniformly distributed, covering the desired spectrum, such as an IR and NIR spectrum. Single location LUS measurements may be performed at different laser vibrometer viewing angles, which may be repeated over a limited sample of body locations. A model may be developed for the angular resolved reflectivity of skin as a function of its attributes (texture, bulk reflectivity, and the like), angle of reflection, and optical wavelength. Laser vibrometer response and variation may be adjusted based upon the resulting measurements and model.

In some configurations, subjects or targets may include a surface enhancement or treatment layer, as described above, and the transmit and/or receive beams can be tuned to the surface treatment layer. In a non-limiting example, the wavelengths used for the transmit and/or receive beams can change depending on the skin surface or the surface treatment optical properties, and the like. LUS arrays may be adaptive to the surface geometry, such as a skin surface, rather than the fixed array geometry found in conventional ultrasound systems. Both image reconstruction with detectors and beamforming in transmission may rely on knowing the surface geometry and localization of the transmit and receive spots, as described above. In some configurations, subject motion is mitigated by repeated measurements to provide for repeated beam parameter updates that compensate for any changes due to motion.

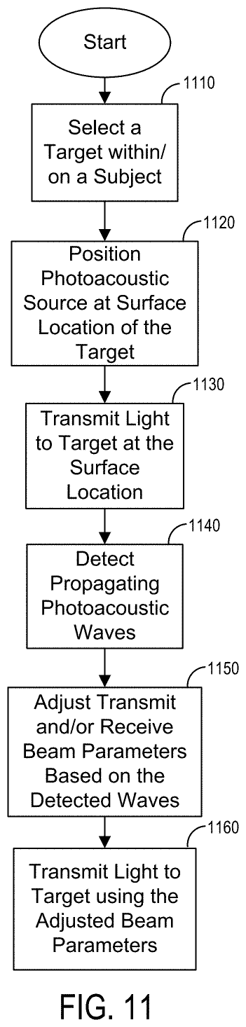

Referring to FIG. 11, a flowchart of non-limiting example steps for generating propagating photoacoustic waves in a subject is shown. A target may be selected within or on a subject at step 1110. A photoacoustic source and detector may be positioned at a surface location over the subject target at step 1120. Light may be transmitted at the target at the surface location at step 1130 using an initial set of beam parameters. Propagating photoacoustic waves may be detected at step 1140 also using an initial set of beam parameters. Adjustments to the transmit and/or receive beam parameters may be made at step 1150 based upon the detected photoacoustic waves. In some configurations, the adjustments may be made to optimize photoacoustic coupling in the subject, to reduce noise, adjust the focusing depth, to optimize time delays, increase signal, address surface reflectivity, address surface orientation, and the like. Light may be re-transmitted to the target using the adjusted beam parameters at step 1160.

In some configurations, an image need not be generated of the subject or target. A map of the subject, a target within the subject, or a property map within the subject may be generated. In non-limiting examples, the map may be a quantitative map, such as a speed of sound map, a density map, an attenuation map, and the like. In a non-limiting example, the map is a speed of sound map. A speed of sound map may report only values for the speed of sound at certain locations and need not be an image generated of the subject.

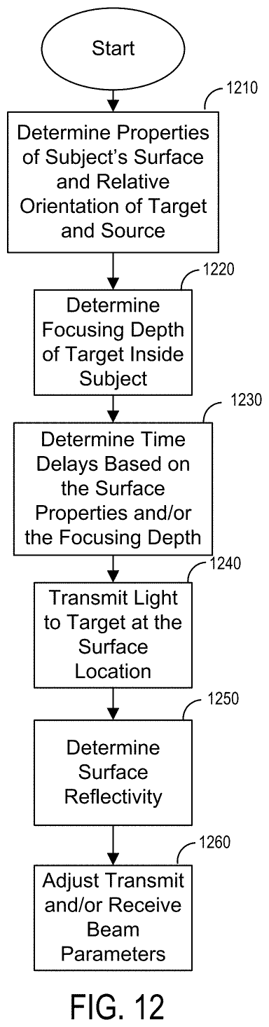

Referring to FIG. 12, another flowchart of non-limiting example steps for generating propagating photoacoustic waves in a subject is shown. Surface properties of a subject and the relative orientation of a target to a photoacoustic excitation source may be determined at step 1210. In non-limiting examples, surface properties may include surface geometry, surface roughness, or surface reflectivity, and the like. A focusing depth for the photoacoustic excitation source for a target inside or on the surface of a subject may be determined at step 1220. Time delays for transmission or detection beams may be determined at step 1230 based on the focusing depth and/or the surface properties of the subject or target. Light may be transmitted to the target at the surface location on the subject at step 1240. Surface reflectivity may be determined at step 1250. Any adjustments to the transmit and/or receive beam parameters may be performed at step 1260. In some configurations, the adjustments may be made to optimize photoacoustic coupling in the subject, to reduce noise, adjust the focusing depth, to optimize time delays, increase signal, address surface reflectivity, address surface orientation, and the like.

The present disclosure has described one or more preferred embodiments, and it should be appreciated that many equivalents, alternatives, variations, and modifications, aside from those expressly stated, are possible and within the scope of the invention.

1. A method for generating ultrasound images of a subject, the method comprising the steps of:
a) selecting a target within the subject and positioning external to the subject a photoacoustic excitation source and a sensor configured to detect vibrations at the surface of the subject created by propagating photoacoustic waves;
b) determining a focusing depth for photoacoustic excitation based upon a depth of the target from a surface of the subject located between the photoacoustic excitation source and the target;
c) determining surface properties of the surface of the subject;
d) determining a relative orientation between a photoacoustic excitation beam from the photoacoustic excitation source and a detection beam from the sensor;
e) determining beam parameters for the photoacoustic excitation beam and the detection beam based on the surface properties, the focusing depth, and the relative orientation;
f) directing the photoacoustic excitation source to transmit the photoacoustic excitation beam, using the beam parameters, onto the surface of the subject to induce the propagating photoacoustic waves; and
g) generating ultrasound images of the target within the subject, using the sensor with the detection beam configured with the beam parameters.

2. The method of claim 1, wherein beam parameters include at least one of time delays, amplitude, size, position, shape, pulse duration, wavelength, polarization or frequency.

3. The method of claim 2, wherein determining beam parameters includes determining time delays based on the surface properties and the focusing depth.

4. The method of claim 2, wherein surface properties includes at least one of geometry, surface roughness, or reflectivity.

5. The method of claim 4, wherein determining a surface geometry includes using at least one of ultrasound, or a camera to image the surface of the subject.

6. The method of claim 4, wherein determining beam parameters includes determining amplitude, size, and position of the photoacoustic excitation and detection beams based on the determined surface reflectivity and the relative orientation between the photoacoustic excitation source and the surface of the subject.

7. The method of claim 4, wherein determining beam parameters includes determining a beam shape, and wherein the beam shape includes a shape determined to optimize optical absorption of the surface of the subject based upon the surface geometry.

8. The method of claim 4, wherein determining beam parameters includes determining a beam shape, and wherein the beam shape includes at least one of a line or a grid based upon the surface geometry.

9. The method of claim 1, wherein determining the relative orientation between the photoacoustic excitation beam and the detection beam includes determining a location of the photoacoustic excitation beam on the surface of the subject, and a location of the detection beam on the surface of the subject.

10. The method of claim 9, wherein determining the relative location of the transmitted photoacoustic excitation beam and the detection beam includes using a point tracking system.

11. The method of claim 10, wherein using the point tracking system includes using at least one of a stereo imaging system, a structured light imaging system, an optical camera, a LIDAR system, time of flight measurements, calibration locations on the surface of the subject, or a 3D surface characterization of the surface of the subject.

12. The method of claim 1, further comprising determining a location of the photoacoustic excitation source and a location of the sensor, and wherein generating ultrasound images includes using the determined locations of the photoacoustic excitation source and the sensor.

13. The method of claim 1, further comprising determining locations on the surface of the subject for a surface enhancement.

14. The method of claim 13, wherein the surface enhancement includes at least one of a gel, gel pad, liquid, reflector, or a material designed to modify at least one of the surface properties of the surface of the subject.

15. The method of claim 13, wherein the surface enhancements include at least one of a hydrogel, glass beads, reflectors, or a combination of thereof.

16. The method of claim 1, wherein the photoacoustic excitation source includes a plurality of excitation sources configured to transmit a plurality of photoacoustic excitation beams, and
wherein the sensor includes a plurality of detectors configured to detect a plurality of detection beams.

17. A system for generating ultrasound images of a subject, the system comprising:
a photoacoustic excitation source positioned external to the subject and configured to transmit a photoacoustic excitation beam onto a surface of a subject to induce propagating photoacoustic waves;
a sensor positioned external to the subject and configured to use a detection beam to detect vibrations at the surface of the subject created by the propagating photoacoustic waves;
a computer system configured to:
i) determine a focusing depth for photoacoustic excitation based upon a depth of a target from the surface of the subject located between the photoacoustic excitation source and the target;
ii) determine surface properties of the surface of the subject;
iii) determine a relative orientation between the photoacoustic excitation beam from the photoacoustic excitation source and the detection beam from the sensor;
iv) determine beam parameters for the photoacoustic excitation beam and the detection beam based on the surface properties, the focusing depth, and the relative orientation;
v) direct the photoacoustic excitation source to transmit the photoacoustic excitation beam, using the beam parameters, onto the surface of the subject to induce the propagating photoacoustic waves; and
vi) generate ultrasound images of the target within the subject, using the sensor with the detection beam configured with the beam parameters.

18. The system of claim 17, wherein beam parameters include at least one of time delays, amplitude, size, position, shape, pulse duration, wavelength, polarization, or frequency.

19. The system of claim 18, wherein the computer system is further configured to determine time delays based on the surface properties and the focusing depth.

20. The system of claim 18, wherein surface properties includes at least one of geometry, surface roughness, or reflectivity.

21. The system of claim 20, wherein the computer system is further configured to determine a surface geometry by using at least one of ultrasound, or a camera to image the surface of the subject.

22. The system of claim 20, wherein the computer system is further configured to determine beam parameters by determining amplitude, size, and position of the photoacoustic excitation and detection beams based on the determined surface reflectivity and the relative orientation between the photoacoustic excitation source and the surface of the subject.

23. The system of claim 20, wherein the computer system is further configured to determine beam parameters by determining a beam shape, and wherein the beam shape includes a shape determined to optimize optical absorption of the surface of the subject based upon the surface geometry.

24. The system of claim 20, wherein the computer system is further configured to determine beam parameters by determining a beam shape, and wherein the beam shape includes at least one of a line or a grid based upon the surface geometry.

25. The system of claim 17, wherein the computer system is further configured to determine the relative orientation between the photoacoustic excitation beam and the detection beam by determining a location of the photoacoustic excitation beam on the surface of the subject, and a location of the detection beam on the surface of the subject.

26. The system of claim 25, wherein determining the relative location of the transmitted photoacoustic excitation beam and the detection beam includes using a point tracking system.

27. The system of claim 26, wherein the point tracking system includes at least one of a stereo imaging system, a structured light imaging system, an optical camera, a LIDAR system, time of flight measurements, calibration locations on the surface of the subject, or a 3D surface characterization of the surface of the subject.

28. The system of claim 17, wherein the computer system is further configured to determine a location of the photoacoustic excitation source and a location of the sensor, and wherein generating ultrasound images includes using the determined locations of the photoacoustic excitation source and the sensor.

29. The system of claim 17, wherein the computer system is further configured to determine locations on the surface of the subject for a surface enhancement.

30. The system of claim 29, wherein the surface enhancement includes at least one of a gel, gel pad, liquid, reflector, or a material designed to modify at least one of the surface properties of the surface of the subject.

31. The system of claim 29, wherein the surface enhancements include at least one of a hydrogel, glass beads, reflectors, or a combination of thereof.

32. The method of claim 17, wherein the photoacoustic excitation source includes a plurality of excitation sources configured to transmit a plurality of photoacoustic excitation beams, and
wherein the sensor includes a plurality of detectors configured to detect a plurality of detection beams.

33. A method for generating ultrasound images of a subject, the method comprising the steps of:
a) determining a focusing depth for photoacoustic excitation of a target based upon a depth of the target from a surface of the subject located between a photoacoustic excitation source, a sensor configured to detect vibrations at the surface of the subject created by propagating photoacoustic waves, and the target;
b) determining surface properties of the surface of the subject;
c) determining a relative orientation between a photoacoustic excitation beam from the photoacoustic excitation source and a detection beam from the sensor;
d) determining initial beam parameters for the photoacoustic excitation beam and the detection beam based on the surface properties, the focusing depth, and the relative orientation;
e) directing the photoacoustic excitation source to transmit the photoacoustic excitation beam, using the initial beam parameters, onto the surface of the subject to induce the propagating photoacoustic waves;
f) determining, using the sensor with the detection beam configured with the initial beam parameters, if the detected vibrations are within a threshold value;
g) updating beam parameters for the photoacoustic excitation beam and the detection beam from the initial beam parameters to adjusted beam parameters by repeating steps a)-f) until the threshold value is achieved; and
h) generating ultrasound images of the target within the subject, using the adjusted beam parameters.

34. A method for generating a map of a subject, the method comprising the steps of:
a) selecting a target within the subject and positioning external to the subject a photoacoustic excitation source and a sensor configured to detect vibrations at the surface of the subject created by propagating photoacoustic waves;
b) directing the photoacoustic excitation source to transmit a photoacoustic excitation beam, using initial beam parameters, onto the surface of the subject to induce the propagating photoacoustic waves;
c) detecting the propagating photoacoustic waves, using the sensor, with a detection beam configured with the initial beam parameters;
d) determining adjusted beam parameters for the photoacoustic excitation beam and the detection beam based on the detected propagating photoacoustic waves
e) directing the photoacoustic excitation source to transmit the photoacoustic excitation beam, using the adjusted beam parameters, onto the surface of the subject to induce the propagating photoacoustic waves;
f) generating a map of the target within the subject, using the sensor, with the detection beam configured with the adjusted beam parameters.

35. The method of claim 34, wherein the map is at least one of a speed of sound map, a density map, or an attenuation map of the subject.
